(19) United States
(12) Patent Application Publication
(10) Pub. No.: US 2019/0029541 A1
(43) Pub. Date: Jan. 31, 2019

(54) FINGER CUFF WITH AN EXPANDABLE COIL TO BE USED IN MEASURING A PATIENT'S BLOOD PRESSURE BY A BLOOD PRESSURE MEASUREMENT SYSTEM

(71) Applicant: Edwards Lifesciences Corporation, Irvine, CA (US)

(72) Inventors: Jason A. Wine, Placentia, CA (US); Siddarth Kamath Shevgoor, Laguna Beach, CA (US); Blake W. Axelrod, Sierra Madre, CA (US)

(21) Appl. No.: 16/029,852

(22) Filed: Jul. 9, 2018

Related U.S. Application Data
(60) Provisional application No. 62/539,067, filed on Jul. 31, 2017.

(51) Int. Cl. A61B 5/022; A61B 5/0225; A61B 5/00

(52) U.S. Cl. CPC A61B 5/02241; A61B 5/02255; A61B 5/6833; A61B 5/6838; A61B 5/6831

(57) ABSTRACT

Disclosed is a finger cuff that may comprise an expandable coil and a bladder. The expandable coil has a finger cavity that includes an LED-PD pair and that is generally smaller than a patient's finger. The expandable coil may include a semi-rigid substrate that is expandable, such that, when the finger cavity of the expandable coil is placed around the patient's finger, the finger cavity and expandable coil expand to surround the patient's finger while the expandable coil provides an approximately constant force to the patient's finger. The bladder is mounted within the finger cavity, such that, when the patient's finger is received and surrounded in the finger cavity of the expandable coil, the patient's finger abuts against the bladder mounted within the finger cavity so that the bladder and the LED-PD pair may be used in measuring the patient's blood pressure by a blood pressure measurement system.

### CROSS-REFERENCE TO RELATED APPLICATIONS

This application claims the benefit of U.S. Provisional Application No. 62/539,067, filed Jul. 31, 2017, which is incorporated herein by reference.

### BACKGROUND

### Field

Embodiments of the invention may relate to a finger cuff with an expandable coil to be used in measuring a patient's blood pressure by a blood pressure measurement system.

### Relevant Background

Volume clamping is a technique for non-invasively measuring blood pressure in which pressure is applied to a subject's finger in such a manner that arterial pressure may be balanced by a time varying pressure to maintain a constant arterial volume. In a properly fitted and calibrated system, the applied time varying pressure is equal to the arterial blood pressure in the finger. The applied time varying pressure may be measured to provide a reading of the patient's arterial blood pressure.

This may be accomplished by a finger cuff that is arranged around a finger of a patient. The finger cuff may include an infrared light source, an infrared sensor, and an inflatable bladder. The infrared light may be sent through the finger in which a finger artery is present. The infrared sensor picks up the infrared light and the amount of infrared light registered by the sensor may be inversely proportional to the artery diameter and indicative of the pressure in the artery.

In the finger cuff implementation, by inflating the bladder in the finger cuff, a pressure is exerted on the finger artery. If the pressure is high enough, it will compress the artery and the amount of light registered by the sensor will increase. The amount of pressure necessary in the inflatable bladder to compress the artery is dependent on the blood pressure. By controlling the pressure of the inflatable bladder such that the diameter of the finger artery is kept constant, the blood pressure may be monitored in very precise detail as the pressure in the inflatable bladder is directly linked to the blood pressure. In a typical present day finger cuff implementation, a volume clamp system is used with the finger cuff. The volume clamp system typically includes a pressure generating system and a regulating system that includes: a pump, a valve, and a pressure sensor in a closed loop feedback system that are used in the measurement of the arterial volume. To accurately measure blood pressure, the feedback loop provides sufficient pressure generating and releasing capabilities to match the pressure oscillations of the subject's blood pressure.

Current finger cuffs are complicated to place on a patient's finger. It is very important that the finger cuff is placed on the patient's finger correctly in order for the blood pressure measurement system to obtain and report correct blood pressure measurement values. In order to obtain a correct attachment of the finger cuff to the finger, the finger cuff needs to be placed on the finger at the correct depth, correct angle, and with the correct tightness. Current finger cuffs are often made from a flexible material that a healthcare provider wraps around the patient's finger and locks into place by simple attachment mechanisms, such as, Velcro. These current types of finger cuffs require that the healthcare provider is able to control all three of these variables (e.g., correct depth, correct angle, and correct tightness), simultaneously, while attaching the finger cuff. Unfortunately, this often results in erroneous placement and tightness of the finger cuff. This erroneous placement and tightness may result in an unsuccessful reading of the patient's blood pressure.

### SUMMARY

Embodiments of the invention may relate to a finger cuff that is connectable to a patient's finger to be used in measuring the patient's blood pressure by a blood pressure measurement system. The finger cuff may comprise an expandable coil and a bladder. The expandable coil has a finger cavity that includes a light emitting diode (LED)—photodiode (PD) pair. The finger cavity of the expandable coil is generally smaller than the patient's finger. The expandable coil may include a semi-rigid substrate that is expandable, such that, when the finger cavity of the expandable coil is placed around a patient's finger, the finger cavity and expandable coil expand to surround the patient's finger while the expandable coil provides an approximately constant force to the patient's finger. The bladder is mounted within the finger cavity, such that, when the patient's finger is received and surrounded in the finger cavity of the expandable coil, the patient's finger abuts against the bladder mounted within the finger cavity so that the bladder and the LED-PD pair may be used in measuring the patient's blood pressure by the blood pressure measurement system.

### BRIEF DESCRIPTION OF THE DRAWINGS

FIG. 1 is a diagram of an environment in which a finger cuff of a blood pressure measurement system may be implemented.

FIGS. 2A-2C are views of an expandable coil of a finger cuff according to embodiments of the invention.

FIGS. 3A-3B are views of different sizes of the expandable coil according to embodiments of the invention.

FIG. 4 is a view of finger cuff with an expandable coil with a fastening mechanism according to embodiments of the invention.

FIGS. 5A-5B are views of a saw-tooth latching mechanism to secure the expandable coil of a finger cuff to a patient's finger according to embodiments of the invention.

FIGS. 6A and 6B are views of a slide lock latching mechanism to secure the expandable coil of a finger cuff to a patient's finger according to embodiments of the invention.

FIG. 7 is a view of a top latching mechanism to secure the expandable coil of a finger cuff to a patient's finger according to embodiments of the invention.

FIG. 8 is a view of a tooth-based latching mechanism to secure the expandable coil of a finger cuff to a patient's finger according to embodiments of the invention.

FIG. 9 is a view of a thumb screw latching mechanism to secure the expandable coil of a finger cuff to a patient's finger according to embodiments of the invention.

FIGS. 10A-10C are views of a finger cuff with an expandable coil with a slit opening according to embodiments of the invention.

FIGS. 11A-11C are views of a finger cuff with an expandable coil with a slit opening and also including a rotatable finger cuff connector according to embodiments of the invention.

### DETAILED DESCRIPTION

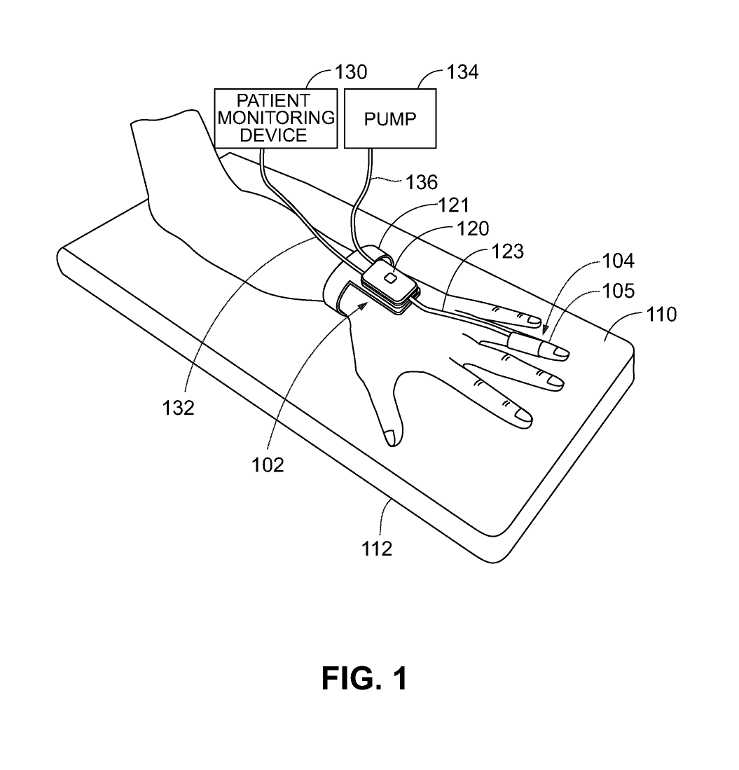

With reference to FIG. 1, an example of an environment in which a finger cuff 104 may be implemented will be described. As an example, a blood pressure measurement system 102 that includes a finger cuff 104 that may be attached to a patient's finger 105 and a blood pressure measurement controller 120 that may be attached to the patient's body (e.g., a patient's wrist or hand) is shown. The blood pressure measurement system 102 may further be connected to a patient monitoring device 130, and, in some embodiments, a pump 134. Further, finger cuff 104 may include a bladder (not shown) and an LED-PD pair (not shown), which are conventional for finger cuffs.

In one embodiment, the blood pressure measurement system 102 may include a pressure measurement controller 120 that includes: a small internal pump, a small internal valve, a pressure sensor, and control circuitry. In this embodiment, the control circuitry may be configured to: control the pneumatic pressure applied by the internal pump to the bladder of the finger cuff 104 to replicate the patient's blood pressure based upon measuring the pleth signal received from the LED-PD pair of the finger cuff 104. Further, the control circuitry may be configured to: control the opening of the internal valve to release pneumatic pressure from the bladder; or the internal valve may simply be an orifice that is not controlled. Additionally, the control circuitry may be configured to: measure the patient's blood pressure by monitoring the pressure of the bladder based upon the input from a pressure sensor, which should be the same as patient's blood pressure, and may display the patient's blood pressure on the patient monitoring device 130.

In another embodiment, a conventional pressure generating and regulating system may be utilized, in which, a pump 134 is located remotely from the body of the patient. In this embodiment, the blood pressure measurement controller 120 receives pneumatic pressure from remote pump 134 through tube 136 and passes on the pneumatic pressure through tube 123 to the bladder of finger cuff 104. Blood pressure measurement device controller 120 may also control the pneumatic pressure (e.g., utilizing a controllable valve) applied to the finger cuff 104 as well as other functions. In this example, the pneumatic pressure applied by the pump 134 to the bladder of finger cuff 104 to replicate the patient's blood pressure based upon measuring the pleth signal received from the LED-PD pair of the finger cuff 104 and measuring the patient's blood pressure by monitoring the pressure of the bladder may be controlled by the blood pressure measurement controller 120 and/or a remote computing device and/or the pump 134 and/or the patient monitoring device 130. In some embodiments, a blood pressure measurement controller 120 is not used at all and there is simply a connection from the tube 123 to finger cuff connector 122 from a remote pump 134 including a remote pressure regulatory system, and all processing for the pressure generating and regulatory system, data processing, and display is performed by a remote computing device.

Continuing with this example, as shown in FIG. 1, a patient's hand may be placed on the face 110 of an arm rest 112 for measuring a patient's blood pressure with the blood pressure measurement system 102. The blood pressure measurement controller 120 of the blood pressure measurement system 102 may be coupled to a bladder of the finger cuff 104 in order to provide pneumatic pressure to the bladder for use in blood pressure measurement. Blood pressure measurement controller 120 may be coupled to the patient monitoring device 130 through a power/data cable 132. Also, in one embodiment, as previously described, in a remote implementation, blood pressure measurement controller 120 may be coupled to a remote pump 134 through tube 136 to receive pneumatic pressure for the bladder of the finger cuff 104. The patient monitoring device 130 may be any type of medical electronic device that may read, collect, process, display, etc., physiological readings/data of a patient including blood pressure, as well as any other suitable physiological patient readings. Accordingly, power/data cable 132 may transmit data to and from patient monitoring device 130 and also may provide power from the patient monitoring device 130 to the blood pressure measurement controller 120 and finger cuff 104.

As can be seen in FIG. 1, in one example, the finger cuff 104 may be attached to a patient's finger 105 and the blood pressure measurement controller 120 may be attached on the patient's hand or wrist with an attachment bracelet 121 that wraps around the patient's wrist or hand. The attachment bracelet 121 may be metal, plastic, Velcro, etc. It should be appreciated that this is just one example of attaching a blood pressure measurement controller 120 and that any suitable way of attaching a blood pressure measurement controller to a patient's body or in close proximity to a patient's body may be utilized and that, in some embodiments, a blood pressure measurement controller 120 may not be used at all. It should further be appreciated that the finger cuff 104 may be connected to a blood pressure measurement controller described herein, or a pressure generating and regulating system of any other kind, such as a conventional pressure generating and regulating system that is located remotely from the body of the patient (e.g., a pump 134 located remotely from a patient). Any kind of pressure generating and regulating system that can be used, including but not limited to the blood pressure measurement controller, may be described simply as a pressure generating and regulating system.

Embodiments of the invention may relate to a finger cuff 104 that is connectable to a patient's finger 105 that may be used in measuring the patient's blood pressure by a blood pressure measurement system utilizing the volume clamp method, as previously described. Aspect of the invention, as will be described, relate to a finger cuff 104 that includes an expandable coil having a finger cavity. The finger cavity may include a light emitting diode (LED)—photo diode (PD) pair and a bladder. The finger cavity of the expandable coil may be smaller than the patient's finger 105. The expandable coil may include a semi-ridged substrate that is expandable, such that, when the finger cavity of the expandable coil is placed around the patient's finger 105, the finger cavity and the expandable coil expand to surround the patient's finger 105 and at the same time the expandable coil provides an approximately constant force to the patient's finger 105. The bladder is mounted within the finger cavity. Therefore, when the patient's finger 105 is received and surrounded in the finger cavity of the expandable coil, the patient's finger 105 abuts against the bladder mounted within the finger cavity such that the bladder and the LED-PD pair are used in measuring the patient's blood pressure by the blood pressure measurement system utilizing the volume clamp method.

It should be noted that it is important that the finger cuff 104 is placed on the patient's finger 105 correctly in order for the blood pressure measurement system to obtain and report correct blood pressure measurement values. In order to obtain a correct attachment of the finger cuff to the finger, the finger cuff needs to be placed on the finger at the correct depth, correct angle, and with the correct tightness. Current types of finger cuffs require that the healthcare provider controls all three of these variables (e.g. correct depth, correct angle, and correct tightness), simultaneously, while attaching the finger cuff. Unfortunately, this often results in erroneous placement and tightness of the finger cuff resulting in potential errors in blood pressure measurement by the blood pressure measurement system.

Embodiments of the invention relate to a finger cuff 104 that automatically controls the tightness of the finger cuff 104 on the finger 105 and provides geometry which makes the correct depth and rotation of the finger cuff 104 when attached to a patient's finger 105 by a healthcare provider more intuitive. This makes the placement and attachment of the finger cuff 104 more simple and accurate. As will be described, finger cuff 104, according to embodiments of the invention, is smaller than the intended finger size (or range of intended finger sizes), which will expand to the patient's finger outer profile when placed on the patient's finger 105. The force resisting expansion will be approximately constant over the allowable expansion range normalizing the tightness of the finger cuff 104 to the patent's finger 105 when installed on the patient. These attributes increase the accuracy of the attachment of the finger cuff 104 and adequate tightness of the finger cuff resulting in improved blood pressure measurement by the blood pressure measurement system.

In particular, aspects of the invention may relate to a finger cuff 104 that is integrated into a semi-rigid substrate. This semi-rigid substrate may be formed/molded into a coiled profile with a nominal diameter that is smaller than the lowest intended finger size. As the coil is forced over larger finger diameters, the coil bends outward and expands to fit the larger finger diameter. Given the intended finger sizes, the geometry and the material selection for the rigid coil substrate may be selected to make the force caused from expansion to be correctly sized and approximately constant. Further, as will be described, a suitable clamping system may be utilized with the semi-rigid substrate coil to lock the coil in place so as to become essentially rigid, allowing the internal bladder to expand into the finger to facilitate the volume clamping method.

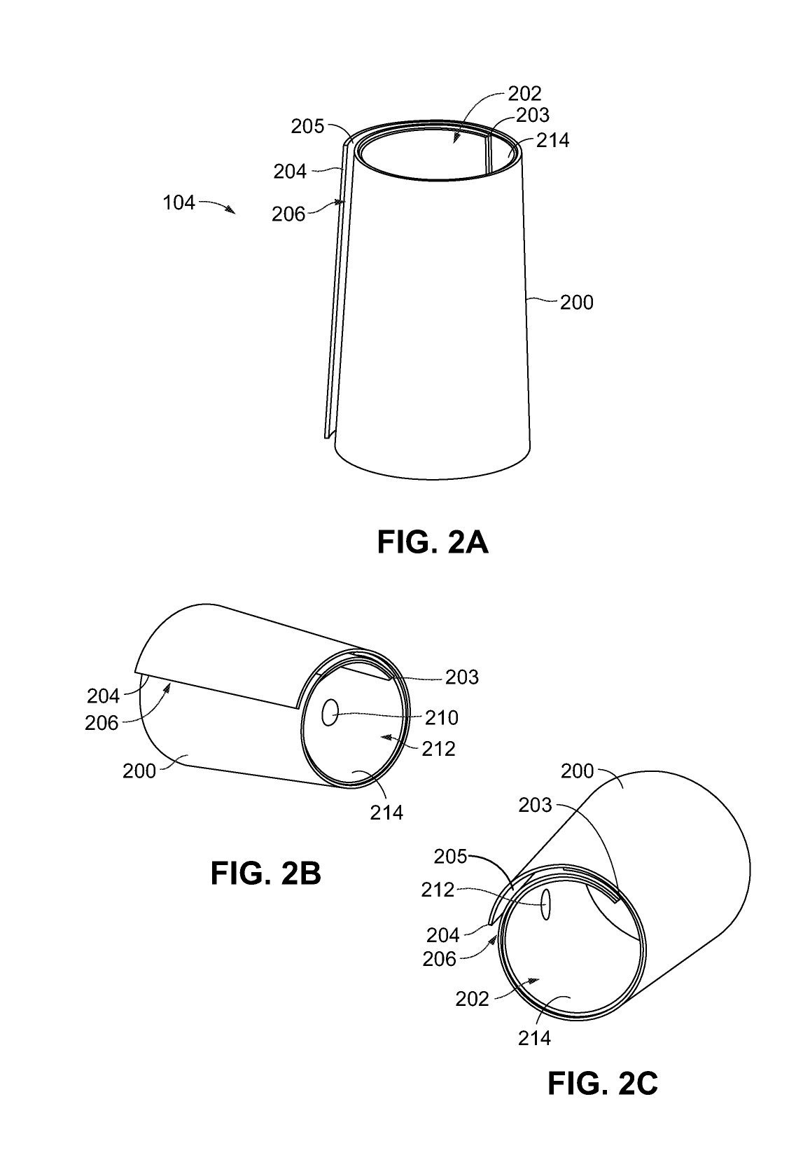

With additional reference to FIGS. 2A-2C, a finger cuff 104 according to embodiments of the present invention that may be connectable to a patient's finger 105 to be used in measuring a patient's blood pressure by a blood pressure measurement system using the volume clamp method, will be described. The finger cuff 104 may include an expandable coil 200 and a bladder 214 that is mounted within the expandable coil 200.

As can be seen in FIGS. 2A-2C, the expandable coil 200 of the finger cuff 104 may be approximately cylindrically shaped and slightly tapers downward in diameter. The expandable coil has a first end 203 and second end 204. The expandable coil 200 forms a finger cavity 202 in which the patient's finger 105 may be placed. In particular, before expansion, the first end 203 of the expandable coil 200 is wrapped inside the finger cavity 202 of the expandable coil 200 and the second end 204 is outside of the finger cavity 202. An opening 206 is formed between the end section 204 and the remainder of the expandable coil 200. The finger cavity 202 of the expandable coil 200 may include a light emitting diode (LED)/photo diode (PD) pair 210 and 212. Further, the bladder 214 may be mountable within the finger cavity 202 of the expandable coil 200. In one embodiment, the bladder 214 may be fully mounted within the interior of the expandable coil 200 within the finger cavity 202 extending from the first end 203 to an end point before the second end 204 leaving an open section 205.

The finger cavity 202 is formed by the expandable coil 200 such that it is generally smaller than a patient's finger, and, is thereafter expandable by the expandable coil 200, to properly fit to the patient's finger. Further, as will be described in more detail hereafter, the expandable coil 200 may be made from a semi-ridged substrate that is expandable to properly fit to the patient's finger. In particular, when the finger cavity 202 of the expandable coil 200 is placed around a patient's finger, the finger cavity 202 and expandable coil 200 expand to surround the patient's finger while the expandable coil 200 provides an approximately constant force to the patient's finger. Further, when the patient's finger is received and surrounded in the finger cavity 202 of the expandable coil 200, the patient's finger abuts against the bladder 214 mounted within the finger cavity 202, such that, the bladder 214 and the LED-PD pair 210 and 212 may be used in measuring the patient's blood pressure by the blood pressure measurements system utilizing the volume clamp method.

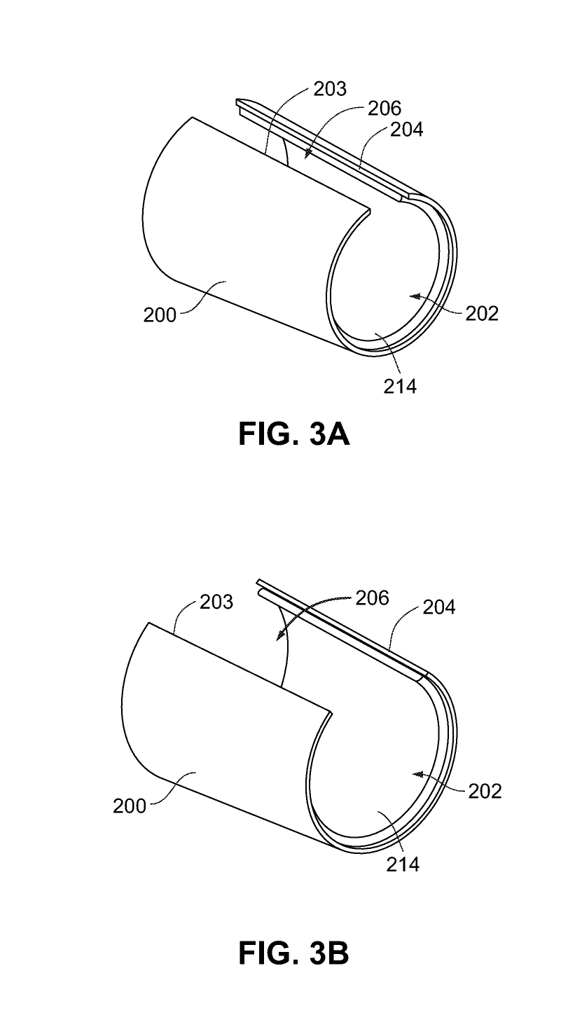

With brief reference to FIG. 3A and FIG. 3B, as can be seen in FIG. 3A and FIG. 3B, the expandable coil 200 and finger cavity 202 are expandable between different sizes—such that the opening 206 between the first end 203 and second end 204 of the expandable coil 200 expands to accommodate a patient's finger—the patient's finger being of possible different sizes (e.g., smaller and larger). For example, FIG. 3A shows an expansion of the expandable coil 200 for a smaller finger size and FIG. 3B shows an expansion of the expandable coil 200 for a larger finger size. In both the smaller and larger expansions, the bladder 214 may abut against the patient's finger to be used to measure the patient's blood pressure by the blood pressure measurement system using the volume clamp method (in conjunction with the LED-PD pair). Thus, the expandable coil 200 expands to allow the finger cavity 202 to expand to surround and accept a patient's finger that may be of variable different sizes.

In order for the coil 200 to be expandable, with an expandable finger cavity 202, such that the expandable coil 200 and expandable finger cavity 202 expand to surround the patient's finger, while at the same time the expandable coil 200 provides an approximately constant force to the patient's finger, a suitable semi-rigid substrate should be utilized to form the expandable coil 200. A wide variety of different materials for a semi-rigid substrates may be utilized. For example, the semi-rigid substrate may include: a polymer; a plastic; a flexible polycarbonate; a rubber; a polyvinyl chloride; a polyethylene terephthalate (PET); a thermoplastic elastomer (TPE); a suitable metallic material; a spring steel; a shaped memory alloy (e.g. a nickel-titanium (nitinol) alloy); etc. It should be appreciated that any type of suitable material to form a semi-rigid substrate to accommodate the previously described functions may be utilized. In particular, the functions of the semi-rigid substrate are to be suitably expandable and to provide an approximately constant force to the patient's finger. Additionally, it should be appreciated that the semi-rigid substrate may include two or more different materials with varying modulus of elasticity (e.g., such as those previously described). Thus, a wide variety of different materials may be combined in differing types of structures (e.g., grid, lattice, truss, helix, stent, etc.) and may be utilized to form a suitable semi-rigid substrate. It should be appreciated that the previous materials are only examples and that at any suitable type of material may be utilized. Further, it should be appreciated that any type of singular material or combinations of combined materials may be utilized to form the semi-rigid substrate in any type of suitable structural formation.

As has been described, in addition to the expandable coil 200 of the finger cuff 104 that expands to surround the patient's finger 105, while simultaneously providing an approximately constant force to the patient's finger, an appropriate latching or fastening mechanism may also be utilized to secure the expandable coil 200 to the patient's finger, such that, expandable coil 200 including the bladder 214 and LED-PD pair 210 and 212 may be used in measuring the patient's blood pressure by the blood pressure measurements system utilizing the volume clamp method. It should be appreciated that a wide variety of different latching, securing, fastening, and clamping mechanisms may be utilized to secure the expandable coil 200 of the finger cuff 104 to the patient's finger 105.

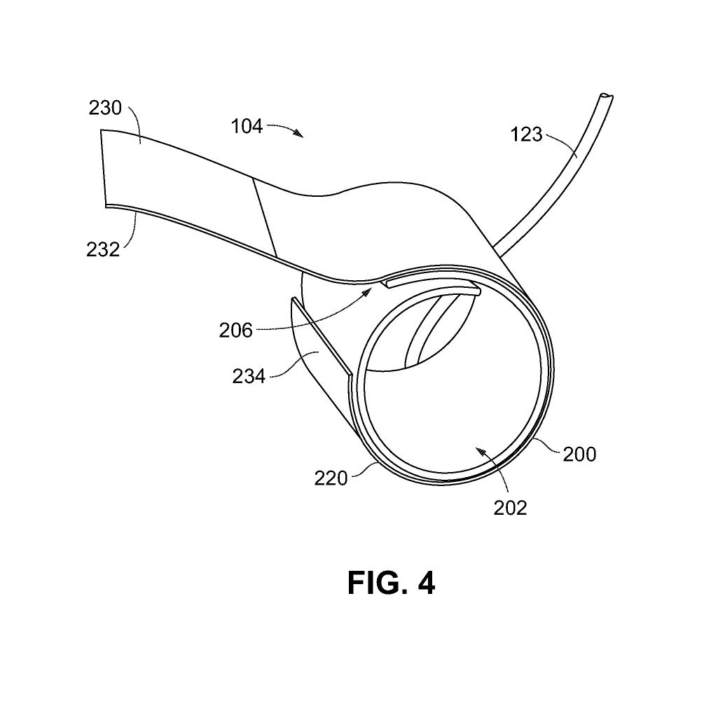

With additional reference to FIG. 4, one type of securing or fastening mechanism that may be utilized as part of the finger cuff 104 with the expandable coil 200 may include an adhesive tape or a Velcro fastener to secure the expandable coil 200 to the patient's finger 105. As can be seen in FIG. 4, the finger cuff 104 includes the expandable coil 200 defining the finger cavity 202 and tube 123 (that is connected to the bladder (not shown)).

Further, a fastening mechanism 220 may be coupled to the outside surface of the expandable coil 200 having a receiving section 234 and a mounting section 230. The fastening mechanism 200 may be attached to the outside surface of the expandable coil 200 and wraps around the expandable coil 200 to provide the mounting section 230 that is connectable to the receiving section 234 to secure the expandable coil 200 to the patient's finger. In particular, the mounting section 230 includes an under side 232 that may include an appropriate fastening mechanism, such as, a Velcro fastener or an adhesive tape that wraps down and mates with the receiving section 234 beneath it to secure the expandable coil 200 to the patient's finger. As an example, a Velcro mechanism (e.g., hook and loop) or reusable adhesive tape configuration may be used for the mounting and receiving sections 230 and 234. In this way, after the patient's finger has been accepted in the finger cavity 220 of the expandable coil 200, which suitably expands to fit the patient's finger, the finger cuff 104 can be firmly connected to the patient's finger by the fastening mechanism 220 (e.g. Velcro, reusable adhesive tape, etc.), in which, the underside mounting section 232 mates with the connection section 234, such that the finger cuff 104 is secured to the patient's finger 105. It should be appreciated that Velcro and reusable adhesive tape are utilized merely as examples, and, that any suitable type of reusable fastening mechanism may be utilized.

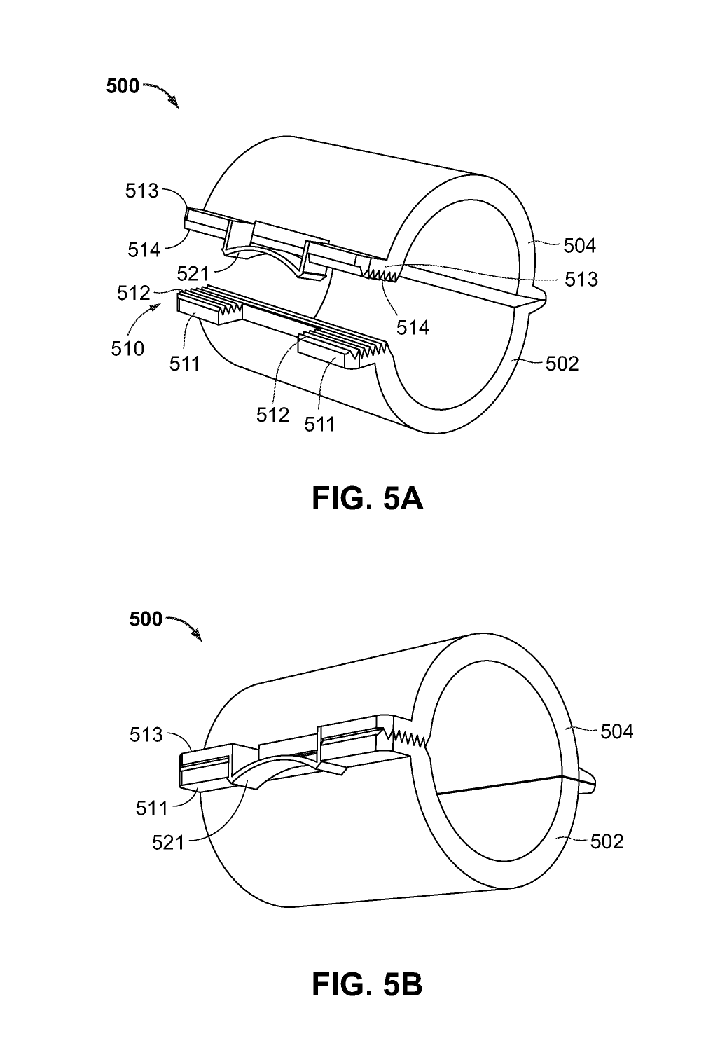

Various other examples of clamping or fastening mechanisms may be utilized to secure the finger cuff 104 to the patient's finger 105. With reference to FIGS. 5A and 5B, a saw-tooth latching mechanism 500 to secure the expandable coil of the finger cuff to the patient's finger may be utilized. The saw-tooth latching mechanism may include a bottom circular latching connector 502 and a top latching connector 504. The bottom and top connectors 502 and 504, as can be seen in FIGS. 5A and 5B, may be rotated open relative to one another and rotated closed to one another. As one example, once the expandable coil has been placed around the patient's finger, the saw-tooth shaped latching mechanism 500 may be opened and then closed around the finger cuff to secure the expandable coil of the finger cuff to the patient's finger.

In order to accomplish this, the saw-tooth latching mechanism 500 includes a pair of opposed top rectangular latching portions 513 on the top latching connector 504 having saw-tooth shaped protrusions 514 and a pair of opposed bottom rectangular latching portions 511 on the bottom latching connector 502 also having saw-tooth shaped protrusions 512 that mate with one another to secure the expandable coil of the finger cuff in place. In particular, when connected together, as shown in FIG. 5B, the saw-tooth shaped protrusions 512 and 514 of the bottom and top portions 511 and 513 mate with one another to connect. Further, an arch-shaped latching mechanism 521 connects between the rectangular bottom portions 511 to further secure the bottom and top connectors 502 and 504 in place. In particular, flanges of the arch-shaped latching mechanism 521 may abut against the rectangular bottom portions 511 to further secure the bottom and top connectors 502 and 504 in place. In this way, the top connector and the bottom connector 504 and 502 of the saw-tooth latching mechanism 500 connect and latch to one another to provide a secure connection for the expandable coil of the finger cuff to the patient's finger. In order to unlock the saw-tooth latching mechanism 500, the healthcare provider simply needs to push up the arch-shaped latching mechanism 521 such that the bottom and top portions 511 and 513 of the bottom and top latching connectors 502 and 504 disengage from one another and the finger can be removed from the finger cuff. It should be appreciated that the saw-tooth latching mechanism 500 may be separate from the expandable coil 200 to secure the finger cuff 104 to the patient's finger 105 or the saw-tooth latching mechanism 500 may be part of or incorporated into the expandable coil 200 to secure the finger cuff 104 to the patient's finger 105.

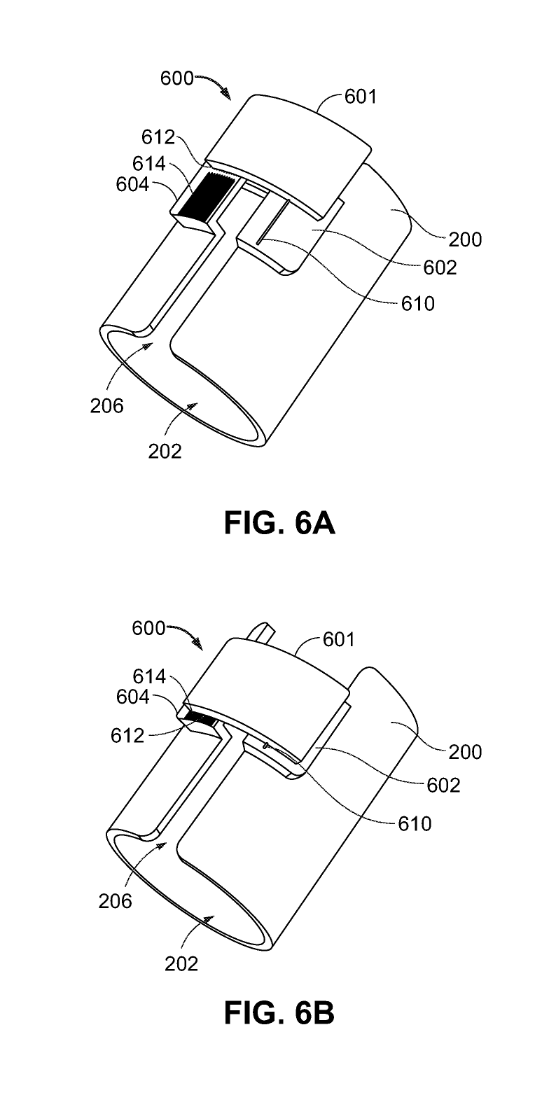

With additional reference to FIGS. 6A and 6B, another type of latching mechanism may be utilized to secure the expandable coil 200 of the finger cuff 104 to the patent's finger 105. In one embodiment, a slide lock mechanism 600 may be coupled to the expandable coil 200 over the expandable coil opening 206. In this embodiment, a first approximately rectangular component 602 having a slot 610 may be mounted to one side of the expandable coil 200 and another approximately rectangular component 604 having a mounting block 614 may mounted to the other side of the expandable coil 200. A slider 601 may be coupled to both components on both sides of the expandable coil 200. The slider 601 may have a sliding component (not shown) that slides within the slot 610 on one side of the expandable coil 200 and a mounting block (not shown) that mounts to the mounting block 614 on the other side of the expandable coil 200. In one embodiment, the mounting block of the slider 601 may include locking teeth (not shown) that slide and engage with the locking teeth of the mounting block 614 on the bottom end. As an example, in FIG. 6A, the expandable coil 200 is unlocked and the patient's finger has been placed in the finger cavity 202 and is ready for locking. Then, in FIG. 6B, the slider 601 has been slid in, such that, the sliding component has slid in the slot 610, while at the same time, the mounting block of the slider 601 has engaged and been mounted with mounting block 614 (e.g., interactive locking teeth) such that the slider 601 has secured the expandable coil 200 of the finger cuff to the patient's finger 105. It should be appreciated that mounting block of the slider and the mounting block of the expandable coil may engage in a variety of different ways (e.g., interacting locking teeth, simply abutting one another, etc.).

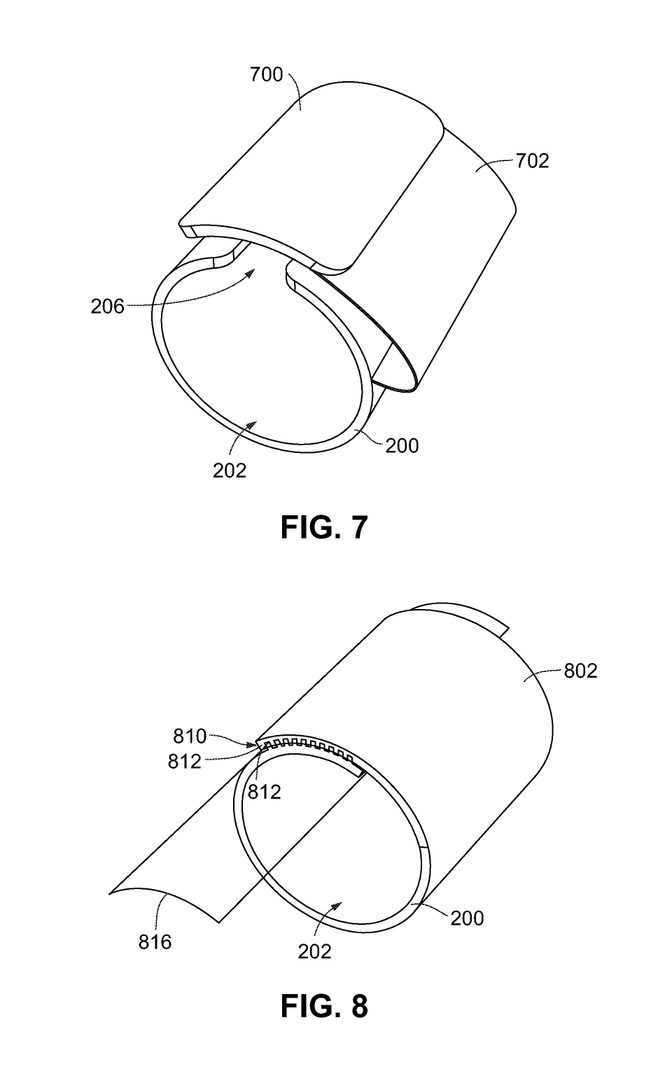

With additional reference to FIG. 7, in one embodiment, the expandable coil 200 of the finger cuff 104 may be secured to the patient's finger by a top latching mechanism 700. The top latching mechanism 700 may be attached to opposite sides on the top of the expandable coil 200 above the opening 206 by suitable attachment mechanisms (e.g., mechanical, Velcro, releasable adhesive, magnetic, etc.), such that, when the patient's finger is placed within the finger cavity 202, the top latching mechanism 700 may be pushed down by a healthcare provider to secure the expandable coil 200 to the patient's finger. On the other hand, when the patient's finger is to be released from the finger cavity 202, the top latching mechanism 700 may be pulled up by a healthcare provider to allow the expandable coil 200 to enlarge such that the patient's finger may be removed. Additionally, a flexible connecting ribbon 702 may be attached to one side of the expandable coil 200 and to the top latching mechanism 700 to ensure that the top latching mechanism 700 is further secured to the expandable coil 200 and to prevent the top latching mechanism 700 from being displaced from the expandable coil 200.

With additional reference to FIG. 8, in one embodiment, an interacting tooth-based latching mechanism 810 may be included with the expandable coil 200 of the finger cuff 104 to secure the expandable coil 200 to the patient's finger 105. The tooth-based latching mechanism 810 may be formed at the beginning portion of the expandable coil 200 with multiple teeth 812 and multiple teeth openings that mate with multiple teeth 812 and multiple teeth openings at the end portion of the expandable coil 200, such that, the multiple teeth interact with one another and may be used to secure the expandable coil 200 to the patient's finger, after the patient's finger is placed in the finger cavity 202. As an example, the patient's finger may be placed in the finger cavity 202 of the expandable coil 200, and the teeth 812 of the tooth-based latching mechanism 810 may interact with one another such that they mate together allowing the expandable coil 200 to expand and fully accept the patient's finger in the finger cavity 202 of the expandable coil 200 and such that they can be pushed together/tightened to secure the expandable coil 200 to the patient's finger. In one embodiment, a pull tab 816 may extend through the interacting tooth-based latching mechanism 810 to keep the teeth from fully engaging until the diameter is set. One the diameter is set, when the patient's finger has been fully accepted in the finger cavity 202 of the expandable coil 200 (and slightly tightened), the pull tab 816 may be pulled out such that the top and bottom teeth 812 completely engage one another. It should be appreciated that the pull tap 816 is optional.

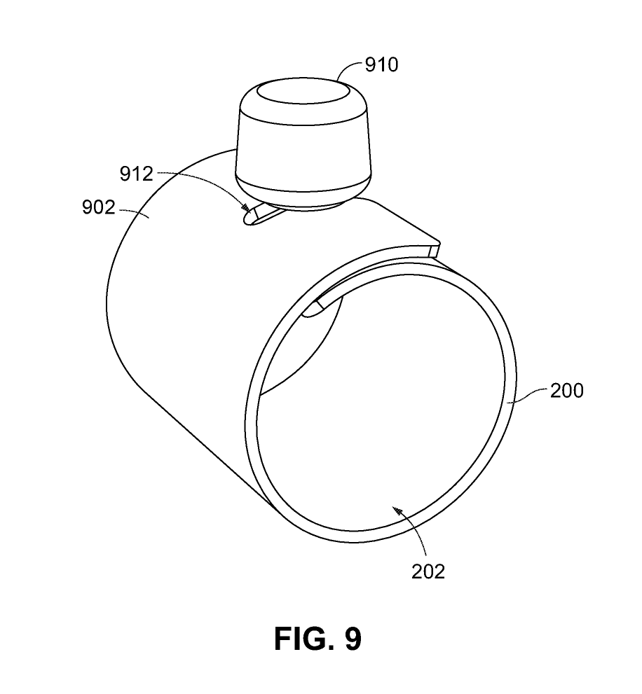

With reference to FIG. 9, a thumb screw latching mechanism may be utilized to secure the expandable coil 200 of the finger cuff 104 to the patient's finger 105. In this embodiment, as can be seen in FIG. 9, the top end portion of the expandable coil 200 may have a slot 912 through which a screw (not shown) attached to the top portion of the beginning section of the expandable coil 200 protrudes. A thumb knob 910 may be utilized to be rotated down upon the screw to a locked position to secure the expandable coil 200 to the patient's finger. Therefore, in this embodiment, after a finger is placed into the finger cavity 202 of the expandable coil 200 and, the expandable coil 200 has expanded, the thumb screw latching mechanism may secure the expandable coil 200 to the patient's finger by the thumb knob 910 being rotated down upon the screw to secure the top portion of the expandable coil 200 to the bottom portion of the expandable coil 200 to secure the expandable coil 200 to the patient's finger. In this way, the cuff diameter is locked into place. On the other hand, the thumb knob 910 may be rotated up away from the screw to unlock and release the expandable coil 200 from the patient's finger. It should be appreciated that the thumb knob with a screw implementation is just one example of a rotatable based fastening or latching mechanism, and that any sort of fastening or latching mechanism may be utilized.

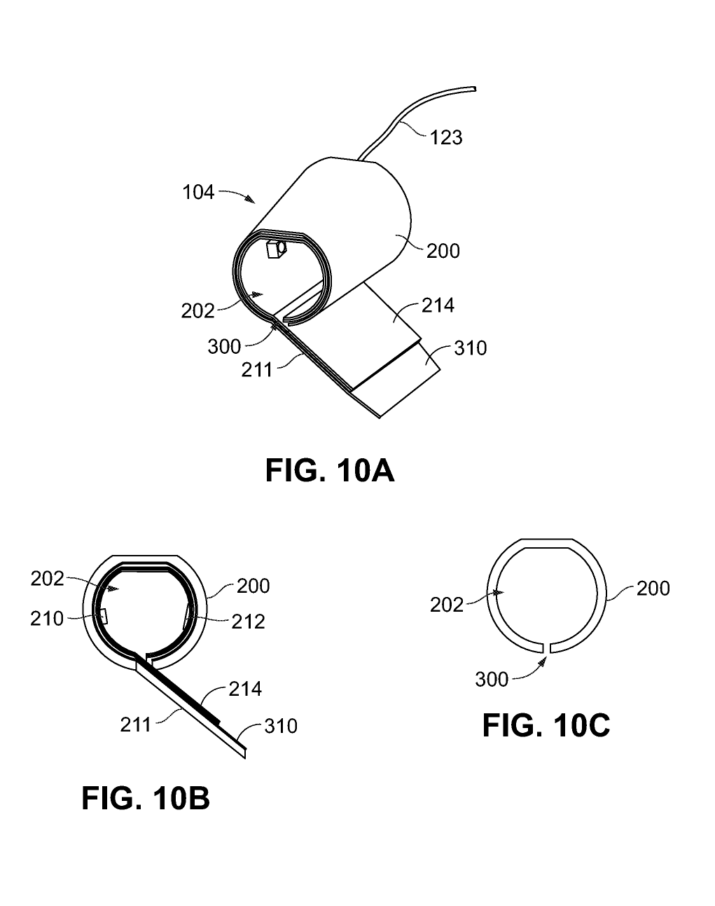

With additional reference to FIGS. 10A-10C, a slight alternative embodiment of the expandable coil 200 of the finger cuff 104 will be described. In this embodiment, the expandable coil 200 similarly has an approximately cylindrical shape that slightly tapers downward in diameter forming a finger cavity 202 in which the patient's finger 105 may be placed, but further includes a split opening 300 at the bottom end. As can be seen in the figures, the finger cavity 202 of the expandable coil 200 includes the LED-PD pair 210 and 212 and the bladder 214. As has been described, the finger cavity 202 of the expandable coil 200 is slightly smaller than a patient's finger such that the expandable coil 200 will expand to accommodate the patient's finger. As has been described previously, the finger cavity 202 of the expandable coil 200 may be placed around a patient's finger such that the finger cavity 202 of the expandable coil 200 and the expandable coil 200 expand to surround the patient's finger while the expandable coil 200 provides an approximately constant force to the patient's finger. Moreover, as has been previously described, the expandable coil 200 may be formed by a semi-ridged substrate.

Additionally, the expandable coil 200 includes a split opening 300 at the bottom end and further includes an extension portion 211 that is attached at the split opening 300 and further extends down below the split opening 300 and below the expandable coil 200. The bladder 214 is fully contained within the finger cavity 202 of the expandable coil 200 and further extends down on extension portion 211 through the split opening 300 and below the expandable coil 200. The extension portion 211 may be made from a flexible material for fastening purposes, as will be described.

In this embodiment, when the patient's finger is received and surrounded in the finger cavity 202 of the expandable coil 200, the patient's finger abuts against the bladder 214 mounted within the finger cavity 202 such that the bladder 214 and the LED-PD pair 201 and 212 may be used in measuring the patient's blood pressure by the blood pressure measurement system, as previously described. Further, as can be seen in FIG. 10A, pneumatic tube 123 may be connected to bladder 214 to pass on the pneumatic pressure to the bladder 214 of finger cuff 104.

Also, in one embodiment, a fastener mechanism may be utilized to secure the expandable coil 200 to the patient's finger. As an example, the fastener mechanism may be a reusable adhesive tape portion 310 that is at the far end of the extension portion 211 below the bladder 214. Once the patient's finger has been placed in the finger cavity 202 of the expandable coil 200, and the finger cavity 202 and the expandable coil 200 have properly expanded to accept the patient's finger, the flexible extension portion 211 may wrapped by a healthcare provider around the exterior of the expandable coil 200 such that the reusable adhesive portion 310 of the flexible extension portion 211 is taped against a section of the exterior of the expandable coil 200. In this way, the reusable adhesive tape portion 310 secures the expandable coil 200 of the finger cuff 104 to the patient's finger 105.

In another embodiment, a Velcro fastener 310 at the far end of the flexible extension portion 211 below the bladder 214 may be used to attach to a mating Velcro portion (e.g., hook and loop) on the exterior of the expandable coil 200 to secure the expandable coil 200 to the patient's finger once the patient's finger has been placed in the finger cavity 202 and the expandable coil 200 and the finger cavity 202 have expanded. In particular, the flexible extension portion 211 may wrapped by a healthcare provider around the exterior of the expandable coil 200 such that Velcro fastener 310 attaches to a mating Velcro portion. In this way, the Velcro fastener 310 secures the expandable coil 200 of the finger cuff 104 to the patient's finger. It should be appreciated that any type of fastener mechanism may be utilized to secure the expandable coil 200 of the finger cuff 104 to the patient's finger 105 and the reusable adhesive tape example and the Velcro fastener example, are merely just examples, and any suitable securing mechanism may be utilized.

The expandable coil 200 of the finger cuff 104 of FIGS. 10A-10C with the slit opening 300 having the expandable finger cavity 202 that expands to surround the patient's finger 105, while at the same time the expandable coil 200 provides an approximately constant force to the patient's finger, may utilize the same types of suitable semi-rigid substrates that have been previously described with respect to the other previously described expandable coils. A wide variety of different materials for a semi-rigid substrates may be utilized. For example, the semi-rigid substrate may include: a polymer; a plastic; a flexible polycarbonate; a rubber; a polyvinyl chloride; a polyethylene terephthalate (PET); a thermoplastic elastomer (TPE); a suitable metallic material; a spring steel; a shaped memory alloy (e.g. a nickel-titanium (nitinol) alloy); etc. It should be appreciated that any type of suitable material to form a semi-rigid substrate to accommodate the previously described functions may be utilized. In particular, the functions of the semi-rigid substrate are to be suitably expandable and to provide an approximately constant force to the patient's finger. Additionally, it should be appreciated that the semi-rigid substrate may include two or more different materials with varying modulus of elasticity (e.g., such as those previously described). Thus, a wide variety of different materials may be combined in differing types of structures (e.g., grid, lattice, truss, helix, stent, etc.) and may be utilized to form a suitable semi-rigid substrate. It should be appreciated that the previous materials are only examples and that at any suitable type of material may be utilized. Further, it should be appreciated that any type of singular material or combinations of combined materials may be utilized to form the semi-rigid substrate in any type of suitable structural formation.

Further, although the previously described expandable coil 200 of FIGS. 10A-10C show a split opening 300 at the bottom end (e.g., 6 o'clock), it should be appreciated that the split opening may be at any suitable position of the expandable coil 200 (e.g., 2 o'clock, 3 o'clock, 9 o'clock, 10 o'clock, 12 o'clock, etc.).

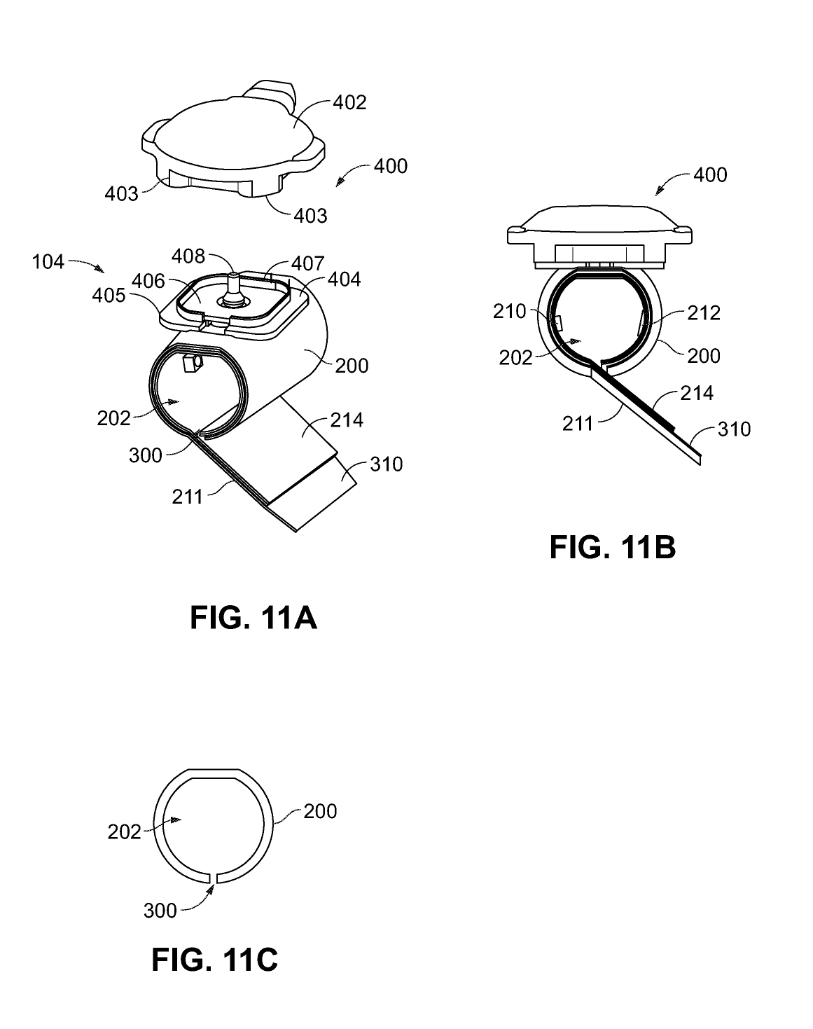

With additional reference to FIGS. 11A-C, a slight alternative embodiment of the finger cuff 104 implementation of FIGS. 10A-C, will be described. The finger cuff 104 with the expandable coil 200 of FIGS. 11A-C is basically exactly the same as that of FIGS. 10A-C, except that the finger cuff of FIGS. 11A-C includes a rotatable finger cuff connector 400. The expandable coil 200 of the finger cuff 104 has an approximately cylindrical shape that slightly tapers downward in diameter forming a finger cavity 202 in which the patient's finger 105 may be placed and includes a split opening 300 at the bottom end. The finger cavity 202 of the expandable coil 200 includes the LED-PD pair 210 and 212 and the bladder 214. As has been described, the finger cavity 202 of the expandable coil 200 is slightly smaller than a patient's finger such that the expandable coil 200 will expand to accommodate the patient's finger. The finger cavity 202 of the expandable coil may be placed around a patient's finger such that the finger cavity 202 of the expandable coil 200 and the expandable coil 200 expand to surround the patient's finger while the expandable coil 200 provides an approximately constant force to the patient's finger. Moreover, the expandable coil 200 may be formed by a semi-ridged substrate. Additionally, the expandable coil 200 includes a split opening 300 at the bottom end and further includes an extension portion 211 that is attached at the split opening 300 and further extends down below the split opening 300 and below the expandable coil 200. The bladder 214 is fully contained within the finger cavity 202 of the expandable coil 200 and further extends down on extension portion 211 through the split opening 300 and below the expandable coil 200. The extension portion 211 may be made from a flexible material for fastening purposes, as will be described.

When the patient's finger is received and surrounded in the finger cavity 202 of the expandable coil 200, the patient's finger abuts against the bladder 214 mounted within the finger cavity 202 such that the bladder 214 and the LED-PD pair 201 and 212 may be used in measuring the patient's blood pressure by the blood pressure measurement system, as previously described. Also, a fastener mechanism may be utilized to secure the expandable coil 200 to the patient's finger. As an example, the fastener mechanism may be a reusable adhesive tape portion 310 that is at the far end of the extension portion 211 below the bladder 214. Once the patient's finger has been placed in the finger cavity 202 of the expandable coil 200, and the finger cavity 202 and the expandable coil 200 have properly expanded to accept the patient's finger, the flexible extension portion 211 may wrapped by a healthcare provider around the exterior of the expandable coil 200 such that the reusable adhesive portion 310 of the flexible extension portion 211 is taped against a section of the exterior of the expandable coil 200. In this way, the reusable adhesive tape portion 310 secures the expandable coil 200 of the finger cuff 104 to the patient's finger. As an alternative, a Velcro fastener 310 at the far end of the flexible extension portion 211 below the bladder 214 may be used to attach to a mating Velcro portion (e.g., hook and loop) on the exterior of the expandable coil 200 to secure the expandable coil 200 to the patient's finger once the patient's finger has been placed in the finger cavity 202 and the expandable coil 200 and the finger cavity 202 have expanded.

Therefore, the expandable finger cuff 200 of FIGS. 11A-C is basically exactly the same as that of FIGS. 10A-C, and the same and similar items will not be repeated for brevity's sake.

The only significant difference between FIGS. 11A-C and FIGS. 10A-C, is that the embodiment of FIGS. 11A-C, utilizes a rotatable finger cuff connector 400 that may be coupled to the finger cuff 104. The rotatable finger cuff connector 400 may be attached to the top portion of the finger cuff 104. The rotatable finger cuff connector 400 may include a finger cuff connector pair 402 and 405. The top finger cuff connector 402 housing may be approximately circular shape with two opposed protrusions for ease of handling, placement, attachment, and rotation by a user to the bottom finger cuff connector 405 and the bottom finger cuff connector 405 may be approximately square shaped and the top and bottom finger cuff connectors 402 and 405 mate together, as will be described. The rotatable finger cuff connector 400 may be used to provide pneumatic pressure to the bladder 214 and a suitable electrical connection to transmit the pleth signal received from the LED-PD pair 210 and 212 of the finger cuff 104 to an appropriate computing device. As has been described, the rotatable finger cuff connector 400 may comprise two halves: a first half 402 that is connected to the pressure generating and regulating system via a pneumatic tube (for the transmission of pneumatic pressure) and electrical wires (for transmitting and receiving electrical signals); and a second half 405 that is fixedly attached to the finger cuff 104 on a square-shaped mounting plate 404. The top first half of finger cuff connector 402 has an open interior and an approximately square-shaped bottom section that contacts the outside sections of mounting plate 404 where they are connected. The interior portion of the top first half of finger cuff connector 402 surrounds an approximately square-shaped mounting section 407 in a manner that provides for a wide variety of possible orientations, as will be described in more detail hereafter. When the first top half 402 and second bottom half 405 are properly connected, electrical and pneumatic connections are arranged within each half of the finger cuff connector pair such that when the first half 402 and the second half 405 of the finger cuff connector pair 400 are properly connected, suitable electrical and pneumatic connections are established between the pressure generating and regulating system and the finger cuff 104.

As an example, properly established electrical connections between the pressure generating and regulating system and the finger cuff 104 may include suitable power, data, and control signal connections between the circuitry of the pressure generating and regulating system and the circuitry of the finger cuff 104 (e.g., the LED-PD pair). To achieve suitable electrical connections, the second half 405 of the finger cuff connector pair may comprise a plurality of electrical connector pads (not shown) on a printed circuit board 406 located within the mounting section 170. When properly connected, in a particular orientation, data from the LED-PD pair of the finger cuff 104 may be transmitted from the connector pads of the printed circuit board 406 through wires to the pressure generating and regulatory system for processing. Further, properly established pneumatic connections between the pressure generating and regulating system and the finger cuff 104 enable a pump of the pressure generating and regulating system to provide pneumatic pressure to the bladder 214 of the finger cuff 104. Pneumatic pressure from a pneumatic tube from the first half 402 may be connected to a tube 408 of the second half 405, which is connected to the bladder 214 of the finger cuff 104. This connection may occur by a connector tube that is rotatably coupled to tube 408 by a suitable rotatable mounting device (e.g., a rotatable seal). In this way, pneumatic pressure may be provided to the bladder 214 of the finger cuff 104 by the pressure generating system through the finger connector pair when the two halves 402 and 405 are connected in any of the possible orientations, to be hereafter described.

The first top half 402 and the second bottom half 405 of the finger cuff connector pair may be connected in four possible orientations. In order to achieve this, mechanical key and magnetic features may be utilized. In particular, the first half 402 of the finger cuff connector pair may be rotated and positioned relative to the fixed second half 405 of the finger cuff connector pair so as to facilitate proper alignment of the connector halves and may be attached to the fixed second half 405 in order to establish suitable electrical and pneumatic connections with the pressure generation and regulatory system. A keying feature in combination with a magnetic feature may be implemented to achieve four possible orientations. In this embodiment, the first top half 402 and the second bottom half 405 of the finger cuff connector pair are connected or mated together. The top first half of finger cuff connector 402 has an open interior and an approximately square-shaped bottom section that contacts the outside sections of mounting plate 404, in which the mounting plate 404 may be formed of a magnetic material. Further, the top first half of finger cuff connector 402 includes four pairs of approximately cylindrically shaped magnets (not shown) that are located approximately at corners 403 of the top first half of finger cuff connector 402. The second bottom half of finger cuff connector 405 has an approximately square-shaped mounting section 407 that includes angled corners. Therefore, as an example, a user may align the first top half 402 with the second bottom half 405, in one of the four previously described orientations, to connect them together. In this connection operation, the interior portion of the top half 402 of the finger cuff connector surrounds the mounting section 407 of the fixed bottom half 405 so that the magnets at the corners 403 of the top half 402 mate with and abut the angled corners of the bottom half 405 to properly align and connect in one of the four different orientation positions. In this way a keying feature is provided. Further, the magnets of the corners 403 abut against the mounting plate 404 and magnetically connect to the magnetic material of the mounting plate 404 such that the first and second halves are magnetically attached to one another (e.g., providing a more secure connection). It should be appreciated that this is just one example, and that a wide variety of orientations may possible, such as: two (90 degrees), six (60 degrees), eight (45 degrees) etc.; dependent upon design considerations.

It should be appreciated that this is just one example of rotatable finger cuff connector that may utilized with the finger cuff connector 104 of FIGS. 11A-11C, and that many other types of rotatable finger cuff connectors may be utilized. As one example, a rotatable finger cuff connector from Applicant's filed co-pending patent application, Ser. No. 62/503,610, which is hereby incorporated by reference, may be utilized.

However, as has been described, except for the use of the rotatable finger cuff connector 402 with the finger cuff 104 of FIGS. 11A-C, the structure and functions of operation of the finger cuff 104, expandable coil 200, bladder 214, LED-PD pair 210 and 212, flexible extension portion 211, fastener 310, etc., is basically exactly the same as that of FIGS. 10A-C, and therefore the operation of these structures will not be repeated for brevity's sake.

The previously described finger cuffs 104 automatically control the tightness of the finger cuff 104 on the finger 105 and provide geometry which makes the correct depth and rotation of the finger cuff when attached to a patient's finger 105 by a health care provider more intuitive. This makes the placement and attachment of the finger cuff 104 more simple and accurate. The previously described finger cuffs 104 are smaller than the intended finger size (or range of intended finger sizes), which expand to the patient's finger outer profile when placed on the patient's finger 105. The force resisting expansion is approximately constant over the allowable expansion range normalizing the tightness of the finger cuff 104 to the patent's finger 105 when installed on the patient. These attributes increase the accuracy of the attachment of the finger cuff and adequate tightness of the finger cuff resulting in improved blood pressure measurement by the blood pressure measurement system.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. A finger cuff connectable to a patient's finger to be used in measuring the patient's blood pressure by a blood pressure measurement system, the finger cuff comprising:
an expandable coil having a finger cavity, the finger cavity including a light emitting diode (LED)—photodiode (PD) pair, the finger cavity of the expandable coil being smaller than the patients finger, the expandable coil including a semi-rigid substrate that is expandable, wherein, when the finger cavity of the expandable coil is placed around a patient's finger, the finger cavity and expandable coil expand to surround the patient's finger while the expandable coil provides an approximately constant force to the patient's finger; and
a bladder mountable within the finger cavity, wherein the patient's finger received and surrounded in the finger cavity of the expandable coil abuts against the bladder mounted within the finger cavity such that the bladder and the LED-PD pair are used in measuring the patient's blood pressure by the blood pressure measurement system.

2. The finger cuff of claim 1, wherein the expandable coil includes an opening that enlarges to allow the expandable coil and finger cavity to expand to surround the patient's finger.

3. The finger cuff of claim 1, wherein the semi-rigid substrate includes a polymer.

4. The finger cuff of claim 1, wherein the semi-rigid substrate includes a flexible polycarbonate.

5. The finger cuff of claim 1, wherein the semi-rigid substrate includes a polyethylene terephthalate (PET).

6. The finger cuff of claim 1, wherein the semi-rigid substrate includes a thermoplastic elastomer (TPE).

7. The finger cuff of claim 1, wherein the semi-rigid substrate includes a metallic material.

8. The finger cuff of claim 1, wherein the semi-rigid substrate includes a shaped memory alloy.

9. The finger cuff of claim 8, wherein the shaped memory alloy includes a nickel-titanium (nitinol) alloy.

10. The finger cuff of claim 1, wherein the semi-rigid substrate includes at least two or more different materials with varying modulus of elasticity.

11. The finger cuff of claim 1, further comprising an adhesive tape to secure the expandable coil to the patient's finger.

12. The finger cuff of claim 1, further comprising a Velcro fastener to secure the expandable coil to the patient's finger.

13. The finger cuff of claim 1, further comprising a saw-tooth shaped latching mechanism to secure the expandable coil to the patient's finger.

14. The finger cuff of claim 1, further comprising a slide lock latching mechanism to secure the expandable coil to the patient's finger.

15. The finger cuff of claim 1, further comprising a top latching mechanism to secure the expandable coil to the patient's finger.

16. The finger cuff of claim 1, further comprising an interacting tooth-based latching mechanism to secure the expandable coil to the patient's finger.

17. The finger cuff of claim 1, further comprising a thumb screw latching mechanism to secure the expandable coil to the patient's finger.

18. A blood pressure measurement system, comprising:
a finger cuff connectable to a patient's finger to be used in measuring the patient's blood pressure, the finger cuff comprising:
an expandable coil having a finger cavity, the finger cavity including a light emitting diode (LED)—photodiode (PD) pair, the finger cavity of the expandable coil being smaller than the patients finger, the expandable coil including a semi-rigid substrate that is expandable, wherein, when the finger cavity of the expandable coil is placed around a patient's finger, the finger cavity and expandable coil expand to surround the patient's finger while the expandable coil provides an approximately constant force to the patient's finger; and
a bladder mountable within the finger cavity, wherein the patient's finger received and surrounded in the finger cavity of the expandable coil abuts against the bladder mounted within the finger cavity such that the bladder and the LED-PD pair are used in measuring the patient's blood pressure by the blood pressure measurement system by a volume clamp method.

19. The blood pressure measurement system of claim 18, wherein the expandable coil includes an opening that enlarges to allow the expandable coil and finger cavity to expand to surround the patient's finger.

20. The blood pressure measurement system of claim 18, wherein the semi-rigid substrate includes a polymer.

21. The blood pressure measurement system of claim 18, wherein the semi-rigid substrate includes a flexible polycarbonate.

22. The blood pressure measurement system of claim 18, wherein the semi-rigid substrate includes a polyethylene terephthalate (PET).

23. The blood pressure measurement system of claim 18, wherein the semi-rigid substrate includes a thermoplastic elastomer (TPE).

24. The blood pressure measurement system of claim 18, wherein the semi-rigid substrate includes a metallic material.

25. The blood pressure measurement system of claim 18, wherein the semi-rigid substrate includes a shaped memory alloy.

26. The blood pressure measurement system of claim 25, wherein the shaped memory alloy includes a nickel-titanium (nitinol) alloy.

27. The blood pressure measurement system of claim 18, wherein the semi-rigid substrate includes at least two or more different materials with varying modulus of elasticity.

28. The blood pressure measurement system of claim 18, further comprising an adhesive tape to secure the expandable coil to the patient's finger.

29. The blood pressure measurement system of claim 18, further comprising a Velcro fastener to secure the expandable coil to the patient's finger.

30. The blood pressure measurement system of claim 18, further comprising a saw-tooth shaped latching mechanism to secure the expandable coil to the patient's finger.

31. The blood pressure measurement system of claim 18, further comprising a slide lock latching mechanism to secure the expandable coil to the patient's finger.

32. The blood pressure measurement system of claim 18, further comprising a top latching mechanism to secure the expandable coil to the patient's finger.

33. The blood pressure measurement system of claim 18, further comprising an interacting tooth-based latching mechanism to secure the expandable coil to the patient's finger.

34. The blood pressure measurement system of claim 18, further comprising a thumb screw latching mechanism to secure the expandable coil to the patient's finger.
